(19) United States
(12) Patent Application Publication
(10) Pub. No.: US 2025/0228512 A1
(43) Pub. Date: Jul. 17, 2025

(54) TOOTH POSITION DETERMINATION AND GENERATION OF 2D RESLICE IMAGES WITH AN ARTIFICIAL NEURAL NETWORK

(71) Applicant: Medicim NV, Mechelen (BE)

(72) Inventors: Bart Goris, Emblem (BE); Pieter Van Leemput, Rumst (BE)

(21) Appl. No.: 18/854,815

(22) PCT Filed: Apr. 5, 2023
(86) PCT No.: PCT/EP2023/059053

§ 371 Date: Oct. 7, 2024

(30) Foreign Application Priority Data
Apr. 8, 2022 (EP) 22167506.9

(51) Int. Cl. A61B 6/51; A61B 6/00; A61B 6/46; G06T 7/00; G06T 7/246; G06T 7/73; G06T 11/00

(52) U.S. Cl. CPC A61B 6/51; A61B 6/461; A61B 6/5223; G06T 7/0012; G06T 7/246; G06T 7/73; G06T 11/008; G06T 2200/04; G06T 2207/10081; G06T 2207/20084; G06T 2207/30036; G06T 2210/41

(57) ABSTRACT

The present disclosure relates to a computer implemented method for automatically generating and displaying a 2D image derived from 3D image data representing a portion of a patient's maxillofacial anatomy. The method comprises the steps of receiving 3D image data comprising a voxel image volume representing the maxillofacial anatomy, the voxel image volume including teeth of the patient and each voxel of the voxel image volume being associated with a radiation intensity value; detecting one or more anatomical landmarks in said voxel image volume using a first artificial neural network; determining a crown center position for each tooth of multiple teeth included in the voxel image volume with the detected anatomical landmarks as reference points and using a second artificial neural network; and generating the 2D image as a reslice image from the 3D image data based on at least one crown center position.

### FIELD OF THE DISCLOSURE

The present disclosure relates to a computer implemented method and data processing system for automatically generating and displaying a 2D image derived from 3D image data representing a portion of a maxillofacial anatomy of a patient using an artificial neural network. It also relates to a method for training the artificial neural network in this respect. The disclosure further relates to a computer program product comprising instructions for automatically generating and displaying a 2D image derived from 3D image data representing a portion of a maxillofacial anatomy of a patient.

### BACKGROUND OF THE DISCLOSURE

The position of anatomical landmarks and views of the maxillofacial anatomy of a patient allows medical professionals such as orthodontists, dentists and dental or maxillofacial surgeons to perform a diagnosis of the patient's situation. This diagnosis enables them to plan, monitor and evaluate an appropriate treatment. The anatomical landmarks are typically indicated by a dental clinician while navigating two- or three-dimensional X-ray images derived from a digitized record of a corresponding anatomical section of the patient. Such a digitized record is for example a 3D rendering of a cone beam computed tomography scan (CBCT-scan) together with orthogonal slices through the scanned volume. Besides indicating the desired landmarks in the 3D data of the patient's anatomy, the dental clinician also requires adequate imaging for an easy and efficient planning, monitoring and evaluation of the desired treatment. However, performing the navigation and annotation tasks manually is a tedious, time consuming task, and requires a substantial amount of training and practice before satisfactory results are achieved.

In particular in case of dental procedures, determining the maxillofacial anatomy in relation to dental procedures in an efficient manner is of high interest. These procedures ask for detailed planning and are frequently performed, i.e. there is a high demand for these procedures. Thus, despite the substantial advancements that have been made in recent years there remains to be a need for enhanced methods and systems that provide efficient support in planning, monitoring and evaluating dental procedures.

### SUMMARY

In view of the above, it is of particular interest for dental clinicians to have systems and methods available that do not burden them with additional tasks when applying the functionality of software. Instead, there is a demand for functionality that allows dental clinicians to dispense of input that they currently have to provide apart from their role in actually performing the essence of their training, i.e. diagnosis or treatment of a patient.

Accordingly, the objective has been to provide methods and systems that take the above into account and in particular decrease the effort and amount of work a dental clinician has to invest before being in a position that allows for deciding on options for a dental treatment in order to meet a patient's needs.

In response thereto, the present disclosure provides a computer implemented method for automatically generating and displaying derived from 3D image data a 2D image representing a portion of a patient's maxillofacial anatomy. The method includes receiving 3D image data comprising a voxel image volume representing the maxillofacial anatomy of the patient, the voxel image volume including teeth of the patient. Each voxel of the voxel image volume is associated with a radiation intensity value. As part of the method, one or more anatomical landmarks in said voxel image volume are detected using a first artificial neural network. Further, a second artificial neural network is used for determining a crown center position for each tooth of multiple teeth included in said voxel image volume with the detected anatomical landmarks as reference points. The 2D image is generated as a reslice image from the voxel image volume based on at least one crown center position.

This computer implemented method allows to automatically generate a 2D image showing a single tooth or multiple teeth from 3D image data received from an imaging means. Thanks to this process, the workload caused for a dental clinician while navigating the 3D image data is significantly reduced. In other words, the dental clinician can start his or her work on a dental treatment with 2D images that have already been generated or that are automatically generated upon a request by the dental clinician. Instead of having to extract the desired views from the image data, the dental clinician has the option to automatically receive these views.

By employing a first artificial neural network for detecting one or more anatomical landmarks, reference points in the voxel image volume of the 3D image data are identified. These reference points are particularly used to identify an image region, where teeth of the patient are or are expected to be present. The detected anatomical landmarks may be used to provide orientation in relation to the 3D image data (e.g. how the maxillofacial anatomy is arranged in the 3D image data or how the maxillofacial anatomy was located and oriented in relation to a scanner, in particular a CT-Scanner).

A second artificial neural network is used to determine a crown center position for each tooth of multiple teeth included in the voxel image volume. The crown center position is in particular a position within the crown volume along the central axis intersecting with the occlusal surface. The crown center position preferably represents the center of a tooth's crown in three dimensions. The crown center positions are determined in the image region comprising teeth of the patient, particularly as indicated through detecting the one or more anatomical landmarks.

Preferably, the second neural network is configured to identify a crown center position dependent on the position of the tooth, in particular the anatomy of the tooth or the tooth type that is expected to be present at this position (e.g. position/anatomy of the tooth as defined in the FDI World Dental Federation notation). In other words, the second neural network is preferably configured or trained to determine each crown center position individually and dependent on tooth type or tooth anatomy.

The first and/or the second artificial neural network is preferably a convolutional neural network. Convolutional neural networks have shown to be particular efficient in analyzing the image data.

A determined crown center position of a tooth in the voxel image volume may serve as a reference point for the 2D image. In particular, the determined crown center position of a tooth is used as a reference point for defining a (e. g. planar or curved) reslice plane through the voxel image volume for generating the reslice image.

The method may further comprise the step of calculating a curved path following a dental arch of the patient's teeth using at least one of said one or more anatomical landmarks as reference point and/or by fitting a curve to said determined crown center positions. Preferably, the curved path is used as a reference for generating the 2D image.

The curved path represents the course or arrangement of the teeth along the dental arch. A dental clinician may use the curved path as a reference for providing a dental restoration for a missing tooth that is well adapted to the dentition of a patient.

The curved path is preferably fitted to pass through or intersect all teeth of the mandible or the maxilla comprised in the voxel image volume.

A curved path may be calculated for each of the mandible side and the maxilla side.

The curved path may be calculated on the basis of one or more anatomical landmarks that have been identified using the first artificial neural network. This allows for providing the curved path as another reference and may assist in determining crown center positions comprised in the voxel image volume and/or in visualizing the 3D data. Further, anatomical landmarks may be used to estimate the course of the curved path of a dental arch if multiple teeth are missing, in particular at an end of a dental arch.

Preferably, the curved path is calculated by fitting a curve to the determined crown center positions. A curved path that uses the crown center positions as reference points is able to reliably and accurately derive the curved path on the basis of the patient's teeth represented in the voxel image volume and/or at least one anatomical landmark. In this way, the accuracy and reliability is also achievable if one or more teeth are missing.

Further, a particularly high reliability and accuracy is achieved if both anatomical landmarks and crown center positions are used as reference points to calculate a curved path following the dental arch of a patient's mandible and/or maxilla.

For example, the method may further comprise the step of detecting the center of the left and/or right condyle head as anatomical landmark by inputting the voxel image volume into the first artificial neural network. If included, the curved path following the dental arch is calculated using the center of the left and/or right condyle head as points or, in particular, as reference points defining the left and right end segments of the curved path.

If the center of the left and/or right condyle head is represented in the voxel image volume of the patient's maxillofacial anatomy, using the at least one condyle head as a reference point allows for a reliable determination of the end(s) of the curved path following the dental arch of a patient.

The reslice image may be a panoramic image generated based on said curved path so that it follows the arch form of the patient's teeth.

Such a reslice image particularly provides an automatically generated overview of the teeth represented in the 3D image data to the dental clinician. It facilitates at least a first assessment of a patient's allover dental situation and allows for a review of the results presented to the dental clinician.

Preferably, this panoramic view is generated based on the curved path. Additionally or alternatively, it may also be generated (directly or indirectly) using the determined crown center positions. The panoramic image may include the dental arch of the maxilla and/or the dental arch of the mandible.

The method may comprise the step of analyzing the 3D image data and in particular the voxel image volume within at least an image region surrounding the determined crown center position of one of the teeth to derive a tooth axis. The tooth axis intersects the coronal crown surface, extends from this intersection towards an apical end of the tooth, and comprises the crown center position determined for the tooth. This step preferably also comprises setting a reslice plane comprising the tooth axis for projecting the reslice image thereon.

Further, deriving the tooth axis preferably comprises, starting from a cross-section of the tooth including the estimated crown center position, the step of tracking a corresponding position in a next adjacent cross-section and proceeding with tracking corresponding positions in adjacent cross-sections along the tooth, in particular in a direction towards the apical side of the tooth, and the step of calculating the tooth axis based on these corresponding positions.

This is a particular fast technique for providing the tooth axis of a tooth and, for this reason, can easily be implemented for each tooth in the patient's image voxel volume. The provision may even be implemented to happen automatically in the background so that the tooth axis is immediately accessible for the dental clinician upon demand.

Once, the corresponding positions in the adjacent cross-sections of the voxel image volume throughout the crown have been tracked (i.e. identified), the tooth axis is preferably calculated based on these corresponding positions using a linear regression.

Preferably, the corresponding positions are tracked in adjacent cross-sections perpendicular to an axis of a coordinate system of the voxel image volume that most closely approximates the direction of the tooth (coronal-apical direction) or the maxillofacial anatomy (cranial-caudal direction). This allows for a particular fast and easy tracking of the corresponding positions in the cross-sections throughout the tooth since tracking is directly based on the 3D image data.

The orientation of the derived tooth axis is preferably calculated relative to the curved path following the dental arch.

Further, the tooth axis represents a useful reference that is preferably included when setting a reslice plane for projecting the reslice image thereon based on the voxel image volume. In other words, the tooth axis particularly facilitates the derivation of a reslice image showing a cross-section of an individual tooth.

For example, the reslice image may be generated and displayed as a cross-sectional reslice image comprising said crown center, wherein the plane of the cross-sectional reslice image is perpendicular to the tooth axis of the tooth.

Accordingly, the tooth axis may serve as a reference for presenting a cross-sectional view of a tooth at the level of the crown center to the dental clinician.

Further, the reslice image may be generated and displayed as a mesial-distal reslice image that comprises the tooth axis and is essentially oriented parallel to the curved path at the position of one of the teeth or a buccal/labial-oral reslice image that comprises the tooth axis and is essentially oriented perpendicular to the curved path at the position of one of the teeth.

Thus, the curved path extending along the dental arch may be used for orienting a reslice plane of a reslice image that represents a cross-sectional view. More specifically, the plane extends in a mesial-distal direction and along the tooth axis, wherein the mesial-distal direction is oriented parallel to the curved path at the position of the tooth (in particular the crown center position or the position on the curved path closest to the crown center position).

Additionally or alternatively, the curved path may be used for orienting a reslice plane of a cross-sectional reslice image so that the plane extends on the one hand in a buccal-oral or labial-oral direction and on the other hand along the tooth axis, wherein the buccal-oral or labial-oral direction is oriented perpendicular to the curved path at the position of the tooth (in particular the crown center position or the position on the tooth axis or the curved path closest to the crown center position).

This significantly reduces the workload of the dental clinician in terms of having to navigate each tooth for creating individual reslice images. Due to this feature, the dental clinician is directly able to select and assess each tooth.

Moreover, starting from the automatically generated reslice image, it is preferably possible for a dental clinician to move and/or rotate the reslice plane the reslice image is projected on. The tooth axis may be used as a reference for moving the reslice plane through the voxel image volume of a tooth. The crown center position may be used as a reference for rotating the reslice plane in the voxel image volume of a tooth.

Before the second artificial network determines the crown center position, the method preferably comprises the steps of defining for each of the teeth assumed to be present in a patient's dentition an image region expected to comprise the representation of this tooth and assigning a label to the image region, wherein the image regions are particularly defined relative to at least one of the anatomical landmarks as reference.

The number and types of teeth assumed to be present in a patient's dentition are preferably based on a complete (i.e. normal) child or adult dentition in accordance with a patient's age. In other words, the system or the method preferably assumes that a patient has a complete dentition. Based on this assumption and on the detected anatomical landmarks, the system assigns above noted image region and preliminary label for each tooth of the complete dentition.

For determining the crown center position of each of the teeth represented in the 3D image data, the second artificial neural network may comprise a module trained to estimate the three components of an offset vector from a position within the voxel image volume towards a predicted crown center position.

This module is preferably trained to determine the crown center position of a tooth on the basis of an assigned (i.e. preliminary) tooth label. In other words, the module may be trained to determine the crown center position of each tooth according to a tooth label of this tooth.

In particular, the second artificial neural network may be trained to estimate offset vectors from multiple positions within the voxel image volume, where a tooth of the patient should be located, in particular an image region in the voxel image volume of the tooth that may have been assigned with a tooth label. Such a configuration results in generating a plurality of crown center predictions within each of said image regions using the second neural network. All crown center predictions determined for a given image region may be marked with the tooth label of said image region or the tooth label of a crown center prediction as determined by the second artificial neural network.

If multiple crown center predictions for an image region have been generated, the (most likely) crown center position may be determined by clustering said crown center predictions, wherein an initial tooth label is assigned to the determined crown center position based on the tooth labels of the clustered crown center predictions.

The initial tooth labels of the determined crown center positions that have been assigned based on the original tooth labels under the assumption of a complete dentition may then be revised on the basis of an analysis of the relative positions of the determined crown center positions. Preferably, the relative positions of the determined crown center positions are compared to the crown center positions in a model dentition. Preferably said model dentition comprises for each tooth type an average crown center position obtained from a plurality of reference dentitions. This allows for assigning revised tooth labels as tooth labels that take differences to a complete dentition into account.

Thus, the dental clinician does not have to assign tooth labels manually but is already presented with tooth labels. Nonetheless, manual revision by a dental profession may well be implemented as an option.

The method may further comprise a step of deriving a virtual crown center position and an associated virtual tooth label for any tooth missing in the 3D image data of the patient from said determined crown center positions and assigned tooth labels.

In other words, on the basis of the determined crown center positions and the revised tooth labels assigned thereto, any tooth missing in the patient's dentition may be identified. In particular, if the determined crown center positions, e.g. along a curved path following the dental arch of a jawbone (maxilla or mandible), and the tooth labels indicate a gap, it is determined depending thereon that one or more teeth are missing. The position of a virtual crown center may be estimated on the basis of the positions of the teeth that have been identified in the voxel image volume as being present and the type of tooth as designated by the virtual tooth label. Another source of input that may be used in deriving a virtual crown center position is above-noted curved path (if calculated).

Accordingly, the overview presented to a dental clinician is able to automatically provide a full overview over the dental situation of a patient. This may, for example, facilitate a manual revision of the tooth label by a dental clinician, if necessary.

Further, the method may comprise the step of displaying a tooth chart generated using the detected crown center positions and associated tooth labels. The tooth chart may be a personalized chart indicating missing teeth and reflecting the relative arrangement of the patient's teeth based on the distances between the detected crown center positions. Preferably, such a tooth chart comprises selectable icons denoting teeth represented in the 3D image data and/or any tooth missing in the 3D image data, wherein upon selection of one of the icons a 2D image of the tooth position of the selected icon is displayed and includes at least a reslice image.

Preferably, the 2D image includes more than one reslice image and, in particular a mesial-distal (coronal), a buccal/labial-oral or labial-oral (sagittal), and/or cross-sectional (transverse) reslice image. These reslice images may be generated as described above.

Upon selection of a tooth chart icon, displaying the 2D image including at least one reslice image allows for a dental clinician to assess each tooth represented in the voxel image volume quickly and efficiently since the at least one reslice image is automatically generated.

The reslice image may be generated in advance or upon selecting the icon of a tooth in the tooth chart.

The method may further comprise displaying the 3D image data and displaying a marker at the position of each determined crown center position and/or each virtual crown center position.

The 3D image data is preferably displayed as a 3-dimensional representation of the jawbone and the teeth. Such a display of the data allows the dental clinician to assess the dental situation of a patient from different angles and/or magnifications according to the preference of the clinician. Thus, the clinician is preferably able to manipulate the display after it has been automatically generated.

The method preferably comprises the step of extrapolating a virtual tooth axis for the at least one missing tooth, wherein the virtual tooth axis comprises the virtual crown center position of the missing tooth, and the step of calculating a direction of the virtual tooth axis based on a local direction of the curved path following the dental arch and/or the direction of a tooth axis of at least one neighboring tooth.

The direction or angle of the virtual tooth axis is preferably calculated relative to the curved path following the dental arch. In particular in this case, the direction of the tooth axis may also be based on the local direction of the curved path. For example, the local direction and the curved path may be used to identify a lingual, buccal, or labial direction and based thereon an apical-coronal direction of the virtual tooth may be derived for defining the virtual tooth axis. Additionally or alternatively, the virtual tooth axis may be based on the direction of a tooth axis of one neighboring tooth, preferably two neighboring teeth. One neighboring tooth may particularly be used as a basis if more than one tooth is missing or if the missing tooth is at the end of a patient's dental arch.

Providing a virtual tooth axis provides a dental clinician at least with a good starting point for planning a restoration and the direction of insertion of an implant or implants supporting the restoration.

Calculating the curved path following the dental arch of the patient's teeth may comprise fitting the curve of the path to both the determined crown center positions of the teeth represented in the 3D image data and each virtual crown center position.

The method may comprise the step of generating and displaying a reslice image of a reslice plane comprising the virtual tooth axis of the missing tooth.

Such a reslice image, for instance a mesial-distal, buccal-oral or labial-oral reslice, may serve for illustrating an implantation site for a dental implant and the direct surroundings of a restoration to be envisaged. It particularly allows a dental clinician to check the usability of the virtual tooth axis as an axis of implantation against these surroundings. Accordingly, it helps to avoid problems during the treatment of the patient by an enhanced planning without causing more functionality workload for the dental clinician.

The 3D image data may be displayed together with a displayed line corresponding to the tooth axis for each crown center position and/or a line corresponding to the virtual tooth axis for each virtual crown center position.

In general, the method and feature of above provide the dental clinician with an enhanced functionality for diagnosing a dental condition, planning, executing, and/or reviewing a treatment without increasing the workload for the clinician by an increase in interaction with the computer implemented method to make use of this functionality. Quite to the contrary, this method replaces a significant part of previous interactions, in particular in terms of having to navigate the 3D image data for imaging.

Thus, the present disclosure particularly provides a method for generating 2D reslice images from volumetric image data in a substantially user-independent and different manner. The existing user-directed workflows typically required a user to find references within the image data, such as tooth position indicators tooth axes or dental curves for an upper and/or lower jaw. However, identifying these references is a tedious task that involves scrolling through various views/slices of image data and is prone to intra- and interindividual variability. Further, if references to be identified in the generation of 2D reslice images were not clearly identifiable anatomic landmarks, their indication particularly tended to vary based on a user's experience as well as personal preferences. It is also noted that already the selection of views/slices of the volumetric data by a user for indicating such references inherently affects the outcome and input for subsequent treatment planning execution and review.

The method of the present disclosure has the advantage that it is user-independent and allows for a uniform and consistent generation of 2D reslice images without interference. Thus, when reviewing reslice images generated according to the present disclosure, a clinician can be assured that the reslice images are the result of an objective workflow with less mistakes and does not have to take into account whether the pre-processing of the volumetric data was performed by the clinician, a colleague, or an assistant. This consistency of reslice image generation is particularly relevant and enhances results when comparing reslice images from subsequent volumetric image data of a same patient at different points in time.

Regarding panoramic reslice images, the method of the present disclosure utilizes said detected crown center positions for fitting dental curves to the upper and lower jaw. This approach provides as particular advantage that it generates a curved reslice plane through the voxel image volume suitable for projecting a panoramic reslice image right away, wherein said curved reslice plane passes through substantially all tooth volumes of the imaged teeth. In contrast, user-directed workflows generally require repetitive adjustments of dental curves in order to obtain a panoramic reslice plane with such a desired path.

### BRIEF DESCRIPTION OF THE DRAWINGS

The following figures illustrate preferred embodiments of the present invention. These embodiments are not to be construed as limiting but merely for enhancing the understanding of the invention in context with the following description. In these figures, same reference signs refer to features throughout the drawings that have the same or an equivalent function and/or structure. It is to be noted that a repetitive description of these components is generally omitted for reasons of conciseness. Concerning the figures:

FIG. 1 is a flow chart illustrating a series of steps performed by the computer implemented method of the present disclosure for generating a 2D image from 3D image data of a patient's dento-maxillofacial anatomy;

FIG. 2 is a flow chart illustrating a series of steps performed by the computer implemented method of the present disclosure for detecting anatomical landmarks in a voxel image volume using a first neural network;

FIG. 3 is a schematic overview of a first neural network for detecting an anatomical landmark that may be implemented in the method illustrated in FIG. 2;

FIG. 4 illustrates three occlusal landmarks that have been detected by a first neural network and are initially used in the method for determining crown center positions;

FIG. 5 is a flow chart for illustrating steps of the computer implemented method to determine crown center positions using a second neural network;

FIG. 6 depicts two volume renderings of a patient's dento-maxillofacial image data from different directions;

FIG. 7 illustrates a spatial order of cluster centers relative to the center point c;

FIG. 8 illustrates two exemplary visualizations of a lower jaw extracted from 3D image data, wherein crown center positions are indicated with dots and the lines of the detected teeth show the estimated tooth axes;

FIG. 9 illustrates an automatically generated panoramic image on the basis of detected crown center positions, wherein (a) displays a panoramic image generated using a dental curve determined for a lower jaw and (b) depicts a panoramic image generated using a dental curve determined for an upper jaw;

FIG. 10 represents exemplary screenshots showing windows respectively displaying a lingual-buccal, mesial-distal and axial reslice of a tooth position automatically derived from volumetric 3D image data, wherein the upper right part of the screenshots shows tooth chart windows for selecting and displaying reslice images of a tooth, such as (a) reslice images for tooth position 23 of an adult patient and (b) reslice images for tooth position 54 of a child patient.

### DETAILED DESCRIPTION

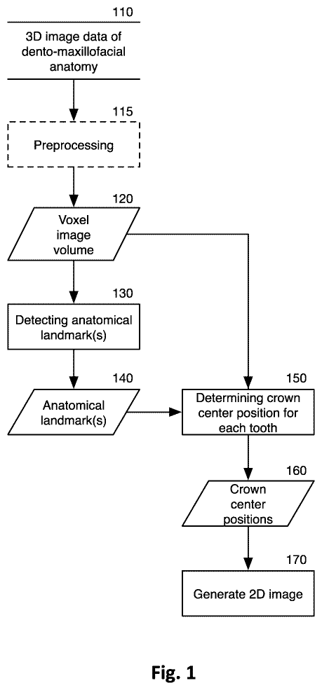

The flow chart of FIG. 1 provides an overview of a computer-implemented method according to the present disclosure. Under reference to FIG. 1, the general method of the present disclosure will be discussed before providing more details on options how to realize aspects of this method.

The 3D image data 110 to be processed includes a voxel image volume 120 representing at least a part of a dento-maxillofacial anatomy of a patient. The 3D image data 110 may further contain additional information such as information about the patient, e.g. age, sex etc.

The voxel image volume 120 preferably originates from CT-Data (computer tomography data) or CBCT-Data (cone beam computer tomography data). Accordingly, the voxels of the voxel image volume 120 of the 3D image data 110 represent intensity values, wherein each of the intensity values of the voxels corresponds to a density value of the scanned volume represented by one of these voxels.

Before using the voxel image volume 120 as input, the voxel image volume is preferably subjected to preprocessing 115 (indicated in FIG. 1 by a dashed line) in accordance with the voxel image volumes used for training the neural networks applied to the voxel image volume 120 in steps 130 and 150. As will be discussed in more detail below, preprocessing may involve an intensity normalization step and/or a resizing step.

With or without preprocessing, the voxel image volume 120 is entering step 130 as input. In step 130, at least one anatomical landmark 130 represented in the voxel image volume 120 is detected by employing a first neural network, preferably using a convolutional neural network (CNN), that has previously been trained to predict the position of at least one landmark 130 to be detected. Examples of anatomical landmarks 130 are listed in Table 1.

TABLE 1
Overview of the 3D landmarks for which the first
convolutional neural network is trained.

| Abbrev. | Full name | Description |
|---|---|---|
| N | Nasion | The midpoint of the frontonasal suture. |
| S | Sella | The centre of the hypophyseal fossa. |
| Por(r) | Right porion | The most superior point of the right external acoustic |
| | | meatus. |
| Por(l) | Left porion | The most superior point of the left external acoustic |
| | | meatus. |
| Orb(r) | Right orbitale | The most inferior point of the right infraor- bital rim. |
| Orb(l) | Left orbitale | The most inferior point of the left infraorbital rim. |
| ANS | Anterior nasal spine | The most anterior midpoint of the anterior nasal |
| | | spine of the maxilla. |
| PNS | Posterior nasal spine | The most posterior midpoint of the posterior nasal |
| | | spine of the palatine bone |
| MNOut(r) | Frontal foramen of | The point of the frontal foramen of the right |
| | mandibular nerve | mandibular nerve. |
| MNOut(l) | Frontal foramen of | The point of the frontal foramen of the left |
| | mandibular nerve | mandibular nerve. |
| MNIn(r) | Posterior foramen of | The point of the posterior foramen of the right |
| | mandibular nerve | mandibular nerve. |
| MNIn(l) | Posterior foramen of | The point of the posterior foramen of the left |
| | mandibular nerve | mandibular nerve. |
| UI | Upper incisor | The most mesial point of the tip of the crown of each |
| | | upper central incisor. |
| Occl(r) | Right occlusal landmark | The point on the occlusal plane between last two |
| | | molars on the right side. |
| Occl(l) | Left occlusal landmark | The point on the occlusal plane between last two |
| | | molars on the left side. |
| TMJcenter(r) | Center of the right | The centre point of the right condyle head. |
| | condyle head | |
| TMJcenter(l) | of the right condyle head | The centre point of the left condyle head. |
| Men | Menton | The most inferior midpoint of the chin on the outline |
| | | of the mandibular symphysis. |
| Go(r) | Right gonion | The point at right side of mandibular that is defined by |
| | | dropping a perpendicular from the intersection point of |
| | | the tangent lines to the posterior margin of the |
| | | mandibular vertical ramus and inferior margin of the |
| | | mandibular body or horizontal ramus. |
| Go(l) | Left gonion | The point at left side of mandibular that is defined |
| | | by dropping a perpendicular from the intersection point |
| | | of the tangent lines to the posterior margin of the |
| | | mandibular vertical ramus and inferior margin of the |
| | | mandibular body or horizontal ramus. |
| Zy(r) | Right zygion | The most lateral point on the outline of the right |
| | | zygomatic arch. |
| Zy(l) | Left zygion | The most lateral point on the outline of the left |
| | | zygomatic arch. |
| A | A-Point | The point of maximum concavity in the mid- line of |
| | | the alveolar process of the maxilla. |
| B | B-Point | The point of maximum concavity in the mid- line of |
| | | the alveolar process of the mandible. |
| Pog | Pogonion | The most anterior midpoint of the chin on the |
| | | outline of the mandibular symphysis. |
| Ba | Basion | The most anterior point of the great foramen |
| | | (foramen magnum) |
| Fz(r) | Right frontozygomatic | The most medial and anterior point of the right |
| | | frontozygomatic suture at the level of the lateral |
| | | orbital rim |
| Fz(l) | Left frontozygomatic | The most medial and anterior point of the right |
| | | frontozygomatic suture at the level of the lateral |
| | | orbital rim |

The method further uses the voxel image volume 120 as input for determining a crown center position 160 for each tooth represented in the voxel image volume 120 in step 150. This process relies on a second neural network, in particular a convolutional neural network, that has previously been trained to predict the position of each tooth represented in the voxel image volume 120. In addition to the voxel image volume 120, the process of step 150 has the detected at least one anatomical landmark 130 as input for determining the crown center positions 160.

Finally, the method generates at least a 2D image in step 170 to be presented to a dental clinician. As previously described, the 2D image is basically generated automatically, at least upon request by the user, i.e. the dental clinician, to provide the 2D image without the necessity to manually or interactively navigate a display of the voxel image volume 120 on a screen. Nonetheless, the method may provide tools to the dental clinician for adapting or fine-tuning the position and/or orientation of the 2D image in the voxel image volume 120. In other words, the method may comprise a user interface for input by the dental clinician to modify the orientation and/or position of a 2D slice extending within the voxel image volume 120. This planar or curved 2D slice serves as a surface onto which the 2D image 170 is projected based on the information (intensity, density) of the voxels.

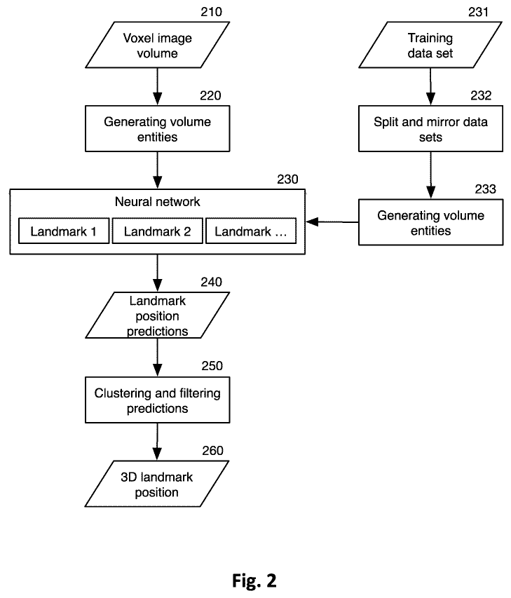

Turning to FIG. 2, the 3D image data 110 may be preprocessed when extracting the voxel image volume 120. In particular, the 3D image data may be normalized and/or rescaled, as will be described in more detail below. This preprocessing preferably adapts the 3D image data 110 to a voxel image volume 120, wherein characteristics of the voxel image volume 120 correspond (e.g. essentially match) to the characteristics of the voxel image volume of data that has been used for training the neural network.

As previously described, the first and/or second neural network is particularly a convolutional neural network albeit other types of neural networks may be used. However, convolutional neural networks have shown to be particularly accurate, efficient, and reliable for image analysis. Nonetheless, in view of this preference, it will also be referred to the neural network as convolutional neural network or “CNN” although other types of neural network may be implemented.

### [Preprocessing—Intensity Normalization]

For example, 3D image data may comprise voxels with intensity values ranging from −1000 for air, about +200 for soft tissue and about +600 for bone tissue. On a screen, these values are preferably displayed using a gray-scale.

Before being used as input for a neural network, the voxel values are preferably rescaled so that the background gray intensity level, the soft tissue gray intensity level (soft tissue value) and/or the hard tissue gray intensity level (hard tissue value) correspond to the levels of the training data for the neural network to be applied to the voxel image volume 120, respectively.

In a first step and to normalize the intensity values I(v)=I(i, j, k) of the original volume of the 3D image data 110, an upper Iₘₐₓ and lower Iₘᵢₙ threshold value may be calculated as follows

$${I}_{min} = max ( \begin{matrix}min \\ v ∈ V\end{matrix} I ( v ) , - 1000 ) \qquad ( 1 )$$

$${I}_{max} = min ( \begin{matrix}max \\ v ∈ V\end{matrix} I ( v ) , 3500 ) \qquad ( 2 )$$

In this example, the intensity values are opted to be bound by values of −1000 and 3500.

Next, these values are used to rescale the original intensity values I(v) of the volume as follows:

$$I ( v ) = s \frac{I ( v ) - {I}_{min}}{{I}_{max} - {I}_{min}} \qquad ( 3 )$$

Here, s is a scaling factor which is initially set to 1. The remaining negative intensity values (3) are set to 0. Thereafter, the scaling factor s is determined for stretching the intensity values so that the soft tissue value or the hard tissue value is scaled to a fixed value. For example, the soft tissue value may be scaled to a fixed value of, for instance, 0.15.

### [Preprocessing—Volume Resampling]

Before or after rescaling the intensity values of the original volume, the volume may be resampled to a fixed voxel size, which preferably corresponds to a voxel size used in the training data. Although the number of voxels may be increased (i.e. to a smaller voxel size), it is advantageous if the resizing step involves subsampling the original volume to a smaller number of voxels (i.e. larger voxel size), preferably without cropping the image volume. By way of reference, it is indicated that CBCT scanning devices imaging the full skull, typically generate a volume image with a voxel size of about 0.4 mm or lower.

In particular depending on the landmark of interest, the volume image may be resampled to a voxel size of about 0.4 mm to 3 mm, preferably to a voxel size of about 0.4 to 2 mm, more preferably to a voxel size of about 0.4 mm to 1 mm, or in particular to a voxel size of approximately 0.4 mm to/or 0.8 mm, if needed in view of the voxel size of the provided 3D image data. For instance, for detecting the frontal foramen of the mandibular nerve, the use of a smaller voxel size, for instance between 2.0 and 0.4 mm, in both the detection and training data is preferred. For the other landmarks listed in Table 1 a comparatively larger voxel size, for instance between, 0.8 and 3.0 mm may be used.

The resizing step may be performed as explained in the following. Considering an original volume has (Nₓ, N_{y}, N_{z}) voxels with a voxel size Δv=(Δx, Δy, Δz), rescaling the volume to a target voxel size of Δv^{(t)}=(Δx^{(t)}, Δy^{(t)}, Δz^{(t)}) means that the number of voxels (Nₓ^{(r)}, N_{y}^{(r)}, N_{z}^{(r)}) in the resampled volume is determined by:

$${N}_{x}^{( r )} = [ {N}_{x} \frac{Δ x}{Δ {x}^{( t )}} ] , {N}_{y}^{( r )} = [ {N}_{y} \frac{Δ y}{Δ {y}^{( t )}} ] and {N}_{z}^{( r )} = [ {N}_{z} \frac{Δ z}{Δ {z}^{( t )}} ] \qquad ( 4 )$$

In (4), “r” denotes “resampled” and “t” denotes “target”. Here, the operator [:] corresponds to integer rounding. Accordingly, the rounding in (4) results in the output volume having an integer number of voxels. Therefore, the voxel size Δv^{(r)}= (Δx^{(r)}, Δy^{(r)}, Δz^{(r)}) of the resampled volume may differ slightly from the target voxel size.

The exact voxel size that matches (4) is given by:

$$Δ {x}^{( r )} = Δ x \frac{{N}_{x}}{{N}_{x}^{( r )}} , Δ {x}^{( r )} = Δ y \frac{{N}_{y}}{{N}_{y}^{( r )}} and Δ {z}^{( r )} = Δ z \frac{{N}_{z}}{{N}_{z}^{( r )}} \qquad ( 5 )$$

Following the calculation of the size of the resampled volume, the volume itself is resampled. The voxel indices v^{(r)}=(i^{(r)}, j^{(r)}, k^{(r)}) of the resampled volume range from 0 to Nₓ^{(r)}−1 in the x-direction, from 0 to N_{y}^{(r)}−1 in the y-direction and from 0 to N_{z}^{(r)}−1 in the Z-direction.

Now, the voxel indices v^{(r)} of the resampled volume are transformed to voxel indices v=(i, j, k) in the original volume. For this, the indices are transformed to a Cartesian position p=(x, y, z) using the formula:

$$p = ( {v}^{( r )} + 0 \text{.5} ) Δ {v}^{( r )} \qquad ( 6 )$$

These distances can be transformed back to non-integer voxel indices in the original volume by using the formula:

$$v = \frac{P}{Δ v} - 0.5 \qquad ( 7 )$$

Because the corresponding volume indices v in the original volume may have non-integer values, a nearest neighbor interpolation may be used to determine the intensity values of the resampled volume.

### [Detecting Landmarks]

The resampled volume data I(v) representing a portion of a patient's skull may be used in a neural network-based step 130 (FIG. 1) for detecting anatomical landmarks, in particular at least one of those listed in Table 1. This step 130 for detecting the at least one anatomical landmark is illustrated in the form of a flowchart in FIG. 2.

### [Detecting Landmarks: Generating Volume Entities]

From the volume I(v), volume entities with a predetermined number of voxels such as 25×25×25 voxels are generated (FIG. 2: step 220). This procedure results in C volume entities, which are centered at voxel indices v=(vₓ, v_{y}, v_{z}) where C∈[1, . . . , C].

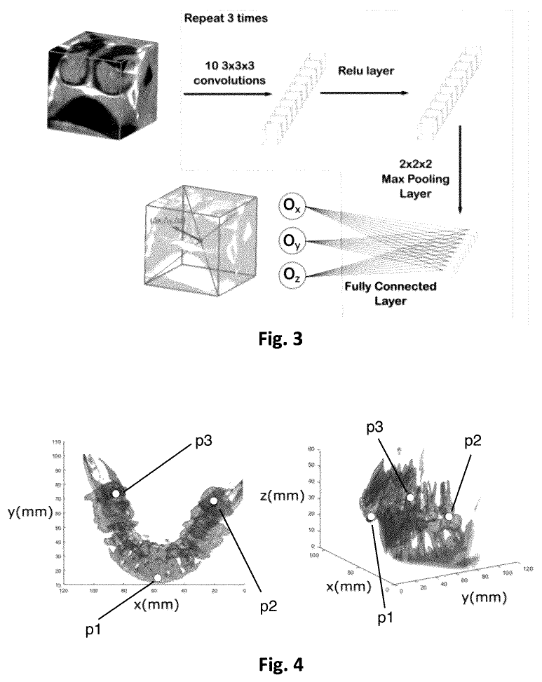

Next, these volume entities are used as input to a neural network, in particular a convolutional neural network (FIG. 2: step 230). An exemplary schematic overview of a preferred 3D convolutional neural network for detecting a landmark is depicted in FIG. 3. For each anatomical landmark to be detected, such a neural network has been trained for the corresponding anatomical landmark before being applied to a patient's voxel image volume. It should be noted that both the first and second neural networks may comprise 3D convolutional neural networks as schematically represented in FIG. 3.

The neural network of FIG. 3 contains three consecutive blocks of convolutional, ReLu and max pooling layers. As illustrated, the network ends with a fully connected layer that has three output values. A volume entity is transmitted through the convolutional layers, ReLu layers and max pooling layers. At the end of the network, the fully connected layer has three outputs corresponding to the components of the offset vector, which estimates the position of a 3D landmark with respect to the volume entity center of a voxel image volume.

The network is trained to estimate the three components of the offset vector v₀ from the volume entity center v towards the voxel indices that the predicted landmark belongs to. Consequently, C different landmark position predictions are obtained at voxel indices (FIG. 2: step 240)

$${v}_{l} = v + {v}_{0} \qquad ( 9 )$$

It is to be noted that these voxel indices may have non-integer values because the components of the estimated offset vector are floats. Next, these resulting non-integer voxel indices are transformed towards Cartesian coordinates p using (6).

### [Detecting Landmarks: Cluster and Filter Predictions]

The following step 250 of FIG. 2 may be implemented for eliminating from the C landmark position predictions those that are considered less accurate than a predetermined threshold. For this, the predictions are initially clustered using a brute force method.

First, the number of inliers for each prediction p are counted, wherein for a selected prediction the inliers are the other predictions, which are located at a distance that is less than a given threshold distance from the selected prediction. Said threshold distance may be set at between 8 and 16 mm, more preferably between 8 and 12, such as at 10 mm. For the prediction having the most inliers, the predicted landmark positions of the inlier volume entities are averaged providing the cluster center k.

Next, the distances from the respective predictions towards this cluster center k are calculated. Predictions at a distance from the cluster center k exceeding said threshold distance are discarded. This results in a prediction list with length \tilde{C}≤C comprising the remaining predictions p for the landmark position. The ratio of the number of inliers \tilde{C} with respect to the number of volume entities C is considered as the probability P for a given landmark.

A high probability means that a trustworthy estimation of the landmark position is obtained, whereas a low probability is indicative of an unreliable estimation of the landmark position. Particularly, estimations are considered unreliable when their probability is lower than between 0.2 and 0.05, preferably between 0.15 and 0.05, for instance below 0.1. An unreliable estimation typically occurs when a searched anatomical landmark is not present in the 3D image data of a patient's dento-maxillofacial anatomy, for example when a skull is only partially scanned.

Some of these detected landmarks may serve for determining crown center positions as will be discussed in more detail below. Further, the detected landmarks may be used to reorient the dento-maxillofacial anatomy and the coordinate system of the 3D image data or voxel image volume relative to each other.

### [Detecting Landmarks: Training]

In this example, the neural networks for detecting the anatomical landmarks, respectively, were trained using a training dataset 231 based on 225 different scans. Here, CBCT scans have been used. However, as discussed above, other CT scanning techniques may be used.

In each scan, the landmarks listed in Table 1 had been indicated. To increase the amount of training data, each CBCT scan was split in half along the x-axis and mirrored with respect to the sagittal plane, where x=0 (FIG. 2: step 232). As a result, 450 symmetrical skull datasets have been obtained.

For each landmark, a network was trained using 150 volume entities for each dataset. Each volume entity was randomly selected at a maximum distance of 12 voxels from the annotated anatomical landmark position. This resulted in a maximum number of 67500 volume entities that are used to train the network.

In this example, all networks were trained in MATLAB (The MathWorks, Inc., Natick, Massachusetts, USA) using the Stochastic Gradient Descent with momentum method. In particular, 100 epochs were used and a mini-batch size of 100 volume entities (an epoch in MATLAB is a measure of the number of times all of the training vectors are used once to update the weights). The order of all volume entities was shuffled before every epoch ensuring that volume entities from various training data were included in each mini-batch. This improved the convergence of the optimization. The initial learning rate equaled 0.0001.

To standardize the training input data, intensity normalization and volume resampling according to predetermined parameters was applied as previously discussed.

### [Determining Crown Center Positions]

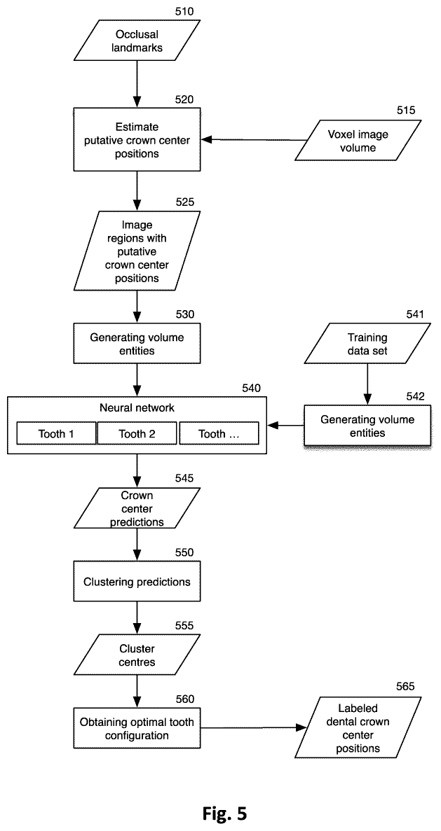

This part of the method determines a crown center position for each tooth based on anatomical landmarks (FIG. 5: step 510) and the voxel image volume (FIG. 5: step 515) using a second neural network (FIG. 1: step 150).

### [Determining Crown Center Positions: Placing Bounding Boxes]

Initially in determining the crown center positions, one or more anatomical landmarks pₒ with o∈[1, 2, 3] that are located on or indicate the occlusal plane that have been detected by the first neural network described above are used (FIG. 2).

In an exemplary embodiment, these occlusal landmarks correspond to the point p₁ in between the tip of the upper incisors, an occlusal landmark p₂ in between the right molars and an occlusal landmark p₃ in between the left molars (see FIG. 4). An example of a transparent isosurface rendering of the dental region of a 3D image dataset where all three landmarks are visualized by dots is shown in FIG. 4.

Nonetheless, other or alternative landmarks may be used as long as they allow for indicating an occlusal plane for the teeth of the maxilla or mandible. This may, for example, be necessary or of advantage if one or more of above-noted occlusal landmarks are not represented in the 3D image data of a patient.

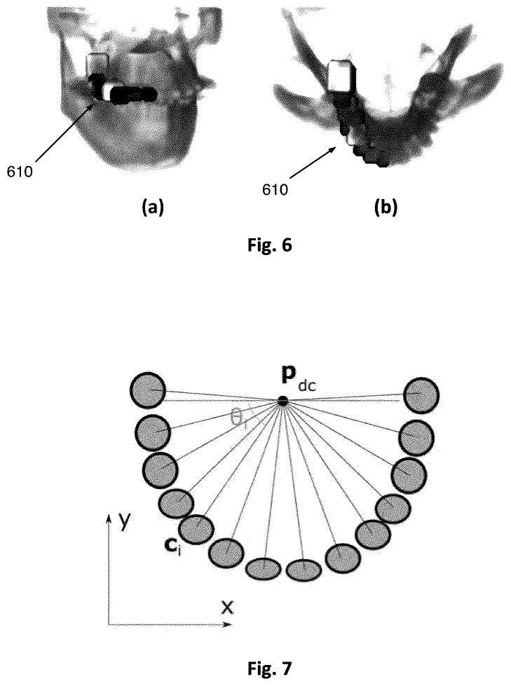

In a next step (FIG. 5: steps 520 and 525), these landmarks are preferably used to estimate bounding boxes 610 centered around putative crown center positions \tilde{t}ⱼ. These bounding boxes 610 serve to indicate the regions in which the respective teeth as present in a normal dentition are assumed to be in the volumetric image. In other words, the bounding boxes 610 are defined by 3D image regions, where the teeth of a normal dentition should be present, respectively. For illustrative purposes, such bounding boxes 610 for tooth numbers 1 to 8 with an exemplary size are visualized in FIG. 6 in a frontal view (a) and an occlusal view (b).

The putative crown center positions \tilde{t}ⱼ are determined using averaged offset vectors āₒⱼ describing the distance and direction from each of said occlusal landmarks to the crown center positions of the respective tooth number positions. The averaged offset vectors āₒⱼ may be determined by averaging the corresponding offset vectors as found for the training data. For an adult dentition, preferably 32 averaged offset vectors āₒⱼ are determined for each occlusal landmark.

For each of said determined occlusal landmarks, the corresponding averaged offset vectors āₒⱼ for the putative crown center positions \tilde{t}ⱼ are estimated. When using two or three occlusal landmarks, two or three independent estimations \hat{t}ⱼ^{(o)} for each of the putative crown center positions are obtained. A final estimated putative crown center position may then be calculated by averaging the corresponding independent estimations.

In case the putative crown center positions \tilde{t}ⱼ were obtained by averaging two or more independent estimations, the respective bounding boxes 610 may be defined by the putative crown center positions ±a multiple (in particular 4 to 6) of their standard deviations (sⱼ), for example using \tilde{t}ⱼ±5sⱼ (FIG. 5: step 525).

In case the putative crown center positions f, are based on single estimations, preset sizes may be used for the bounding boxes 610, in particular sizes so that at least adjacent or multiple bounding boxes 610 overlap. For instance, boxes in a range from 20 mm up to and in particular of 60 mm may be used that are centered around the respective putative crown center positions \tilde{t}ⱼ. Each of these bounding boxes 610 may be labeled with the tooth number of its putative crown center position. For each of these bounding boxes 610, it can be reasonably assumed that it comprises the crown center position of a patient's tooth with the tooth number corresponding to the label of the bounding box 610 unless the imaged patient dentition lacks such tooth.

### [Determining Crown Center Positions: Offset Voting Algorithm]

Within each of the image regions defined by the bounding boxes 610, a second neural network approach is used that is similar or analogous to the offset voting approach applied in landmark detection. In other words, the second neural network also preferably uses an offset voting algorithm for determining crown center predictions (cf. FIG. 3).

Prior to detection, the intensity of the data is preferably normalized and/or the volume is resampled in accordance with the training data as described above. In particular, a resampled volume with a voxel size of at most 2 mm is used, preferably the volume is resampled to a voxel size of about 0.4 mm to 1 mm, more preferably to a voxel size of about 0.4 mm to 0.5 mm, such as to a voxel size of approximately 0.4 mm.

Thereafter, for each possible tooth number, for example, 500 volume entities with a size of preferably 25×25×25 voxels are generated. As the case for the first neural network, a different number of volume entities, in particular in a range from 250 to 650 volume entities may be selected. Also, a volume entity having a different size may be employed, in particular in a range from 15 to 40 voxels along each dimension of the volume entity (FIG. 5: step 530). The voxel indices of the volume entities center are randomly selected from the voxels inside the bounding box 610 with the corresponding tooth number. In other words, voxels from the bounding box 610 are randomly selected, wherein each selected voxel represents the center of a volume entity with the predetermined size for analysis by a neural network as described below.

Next, these volume entities are all processed by a second neural network that is trained to estimate the offset towards the crown center position of a tooth having the tooth number corresponding to that of the bounding box label (FIG. 5: step 540). This yields a number of offset vectors corresponding to the number of volume entities, i.e. in the present example of 500 offset vectors, which, when being added to the volume entity centers, provide a corresponding number of predictions of the crown center position (here: 500 predictions; FIG. 5: step 545).

### [Determining Crown Center Positions: Clustering]

As previously explained, the crown center position detection method yields a corresponding number of crown center predictions for each tooth number. In the example of above, there are 32×500=16000 predictions that all have a corresponding tooth number (32 teeth of a complete adult dentition times 500 volume entities). To obtain the exact coordinates of the crown centers, these predictions are preferably clustered (FIG. 5: step 550).

For clustering, the predictions may be divided into two sets based on the predicted tooth numbers that correspond to the upper and the lower jaw. In other words, the upper and the lower jaw are preferably analyzed separately.

First, clustering step 550 determines for which tooth number a cluster with the most inliers is found. This may be performed using the brute force clustering approach as previously described. In this approach, a predetermined inlier threshold distance (e.g. 1.4 mm) is used, resulting in an averaged crown center cₖ associated with a number of inliers. The number of inliers divided by the number of volume entities (in the present example 500) represents a cluster probability Pₖ.

Within the inlier set (having a size L) associated with said crown center cₖ, the occurrence of each tooth number is counted. Together, these occurrences divided by L form a discrete probability distribution qₖ(j) for the possible tooth number to be assigned to the cluster center cₖ. Then, all said inlier predictions are removed from the list of predictions.

This procedure is repeated for the remaining sets of predictions until 16 cluster centers are detected for each jaw (FIG. 5: step 555).

### [Determining Crown Center Positions: Obtaining the Optimal Tooth Configuration]

The optimal tooth configuration Cₘ may then be determined from the unordered set of 16 cluster center positions cₖ. This tooth configuration labels neighboring crown centers with their respective tooth numbers, wherein the tooth numbers preferably account for possible missing teeth.

First, the clusters with an insufficient number of inliers are considered to relate to missing teeth and are removed. Typically, a threshold ratio in a range of 5% to 15% inliers, in particular of 7% inliers, is used.

Then, the remaining probabilities may be (re) normalized by dividing them by the maximum cluster probability Pₘₐₓ=max(Pₖ: k=1, . . . ,32) resulting in the cluster with the highest probability having a probability equaling 1. All other probabilities are scaled accordingly.

Further, the cluster centers cₖ are split into two sets: one set that relates to teeth that are represented in the image data and one set of cluster centers that relate to teeth that may possibly be represented. The upper and lower probability thresholds used for this discrimination can for instance be set at between 0.6 and 0.4 and between 0.2 and 0.05, such as at 0.5 and 0.1, respectively. Cluster centers with a relative probability below than said lower probability threshold are considered not to relate to represented teeth and are discarded.

Then the optimal tooth configuration C is first searched only considering the cluster centers that are considered to relate to teeth represented in the image data, i.e. the cluster centers with a probability exceeding said upper probability threshold. Such a tooth configuration includes a list of crown centers with their corresponding tooth numbers. In this was, all different possibilities for the K≤16 remaining cluster centers cₖ are investigated to find the best tooth configuration.

Mathematically, there are M possibilities to pick K tooth numbers out of 16:

$$M = ( \begin{matrix}16 \\ k\end{matrix} ) = \frac{16 !}{K ! ( 16 - K ) !} \qquad ( 18 )$$

For example, if 15 clusters centers are found (K=15) in an upper jaw, there are M=16 possible tooth configurations Cₘ^{(K)} with m=1, . . . , M which are presented in table 2.

TABLE 2
Possible tooth configurations when 15 tooth centers
are detected in the upper jaw.

| | | | | | | | | | | | | | | | |
|---|---|---|---|---|---|---|---|---|---|---|---|---|---|---|---|
| C₁^{(K)} | 1 | 2 | 3 | 4 | 5 | 6 | 7 | 8 | 9 | 10 | 11 | 12 | 13 | 14 | 15 |
| C₂^{(K)} | 1 | 2 | 3 | 4 | 5 | 6 | 7 | 8 | 9 | 10 | 11 | 12 | 13 | 14 | 16 |
| C₃^{(K)} | 1 | 2 | 3 | 4 | 5 | 6 | 7 | 8 | 9 | 10 | 11 | 12 | 13 | 15 | 16 |
| C₄^{(K)} | 1 | 2 | 3 | 4 | 5 | 6 | 7 | 8 | 9 | 10 | 11 | 12 | 14 | 15 | 16 |
| C₅^{(K)} | 1 | 2 | 3 | 4 | 5 | 6 | 7 | 8 | 9 | 10 | 11 | 13 | 14 | 15 | 16 |
| C₆^{(K)} | 1 | 2 | 3 | 4 | 5 | 6 | 7 | 8 | 9 | 10 | 12 | 13 | 14 | 15 | 16 |
| C₇^{(K)} | 1 | 2 | 3 | 4 | 5 | 6 | 7 | 8 | 9 | 11 | 12 | 13 | 14 | 15 | 16 |
| C₈^{(K)} | 1 | 2 | 3 | 4 | 5 | 6 | 7 | 8 | 10 | 11 | 12 | 13 | 14 | 15 | 16 |
| C₉^{(K)} | 1 | 2 | 3 | 4 | 5 | 6 | 7 | 9 | 10 | 11 | 12 | 13 | 14 | 15 | 16 |
| C₁₀^{(K)} | 1 | 2 | 3 | 4 | 5 | 6 | 8 | 9 | 10 | 11 | 12 | 13 | 14 | 15 | 16 |
| C₁₁^{(K)} | 1 | 2 | 3 | 4 | 5 | 7 | 8 | 9 | 10 | 11 | 12 | 13 | 14 | 15 | 16 |
| C₁₂^{(K)} | 1 | 2 | 3 | 4 | 6 | 7 | 8 | 9 | 10 | 11 | 12 | 13 | 14 | 15 | 16 |
| C₁₃^{(K)} | 1 | 2 | 3 | 5 | 6 | 7 | 8 | 9 | 10 | 11 | 12 | 13 | 14 | 15 | 16 |
| C₁₄^{(K)} | 1 | 2 | 4 | 5 | 6 | 7 | 8 | 9 | 10 | 11 | 12 | 13 | 14 | 15 | 16 |
| C₁₅^{(K)} | 1 | 3 | 4 | 5 | 6 | 7 | 8 | 9 | 10 | 11 | 12 | 13 | 14 | 15 | 16 |
| C₁₆^{(K)} | 2 | 3 | 4 | 5 | 6 | 7 | 8 | 9 | 10 | 11 | 12 | 13 | 14 | 15 | 16 |

Table 2 lists the upper jaw tooth numbers according to the universal tooth numbering system. In this system, the last right upper molar has tooth number 1 and the last right lower molar has tooth number 32, assuming a complete adult dentition.

Out of these M tooth combinations Cₘ^{(K)} the optimal configuration Cₒₚₜ^{(K)} with the highest configuration probability P(Cₒₚₜ^{(K)}) should be found. This is preferably calculated as the product of the so-called permutation probability Pₚₑᵣₘ(Cₒₚₜ^{(K)}), the distance probability P_{dist}(Cₒₚₜ^{(K)}) and the hypodontia probability P_{hypo}(Cₒₚₜ^{(K)}) as will be explained in more detail below.

$${C}_{opt}^{( K )} = {arg}_{{C}_{m}^{( K )}} max ( P ( {C}_{m}^{( K )} ) ) = {arg}_{{C}_{m}} max ( {P}_{perm} ( {C}_{opt}^{( K )} ) {P}_{dist} ( {C}_{opt}^{( K )} ) {P}_{hypo} ( {C}_{opt}^{( K )} ) ) \qquad ( 20 )$$

Once the optimal tooth configuration is calculated for the set of cluster centers that were considered certain, an additional cluster is selected from the list of possible cluster centers. This cluster center is now added to the configuration, which will then have N+1 cluster centers. For this updated list, the optimal tooth configuration is again calculated using (20).

If the total probability P(Cₒₚₜ^{(K+1)}) of this configuration is higher than, for example, 0.7 P(Cₒₚₜ^{(K)}), the possible cluster center is considered as being a valid crown center and is added to the list of certain teeth.

In a next step, the procedure is repeated and the following possible tooth cluster is investigated until the calculated configuration probability drops below, for example, 0.7 times the previous best configuration probability for all possible additional cluster centers.

The optimal configuration Cₒₚₜ^{(K)} thus assigns a specific tooth label j to each cluster center cₖ, resulting in the labeled dental crown positions {tⱼ}={cₖ} (FIG. 5: step 565). As already noted above, this procedure is preferably performed separately for the upper and lower jaw based on the separation of the predictions in relation to the upper and lower jaw.

### Sorting the Cluster Centers.

To calculate the probabilities of the different configurations, all crown centers are preferably ordered along the dental arch in space (see FIG. 7). For this, the center position Pac of the occlusal dental curve may be roughly estimated or obtained by adding a fixed offset to the most reliable landmark from the occlusal landmarks.

Then, the crown centers are ordered by calculating the angles θᵢ between the vector connecting the cluster centers cₖ and the dental curve center P_{dc} with the x-axis (see default world axis configuration as depicted in FIG. 7). In this example, the cluster centers of the upper jaw are sorted according to increasing angles and the cluster centers of the lower jaw according to decreasing angles θᵢ.

$${θ}_{i} = a tan 2 ( {c}_{k}^{( x )} - {p}_{dc}^{( x )} , - ( {c}_{k}^{( y )} - {p}_{dc}^{( x )} ) ) \qquad ( 21 )$$

### Permutation Probability.

As previously described, every cluster center cₖ has a discrete probability distribution qₖ(j) that aggregates the discrete probabilities of the kth cluster having a specific tooth number j, where j∈[1, . . . , 32]. The permutation probability Pₚₑᵣₘ(Cₘ^{(k)}) of a specific tooth configuration Cₘ^{(k)} may be obtained by multiplying the individual probability distributions of all teeth that are present in the tooth configuration. If there are N tooth clusters that are present in the configuration Cₘ^{(k)} containing the tooth number j, its permutation probability is given by:

$${P}_{perm} ( {C}_{m}^{( k )} ) = {( {\prod }_{k = 1}^{K} {q}_{k} ( j ) )}^{1 / K} \qquad ( 22 )$$

Here, the power 1/K is used to make probabilities comparable for configurations with a different number of cluster centers.

### Distance Probability.

Preferably, the Euclidean distances d_{k,k+1} between neighboring cluster centers are calculated for each configuration with K ordered clusters centers cₖ. These distances are then compared to the mesio-distal averaged distances d̅_{k,k+1} obtained by averaging the distances between neighboring teeth centers in the training data (taking into account missing teeth and hypodontia).

The differences between the various d_{k,k+1} and d̅_{k,k+1} give an indication of how reliable the mesio-distal averaged distances d̅_{k,k+1} obtained by averaging the distances between neighboring teeth centers in the training data (taking into account missing teeth and hypodontia) is in a specific tooth configuration. The total distance probability is defined as:

$${P}_{dist} ( {C}_{m}^{( K )} ) = {( {\prod }_{k = 1}^{K - 1} exp ( \frac{{( {α}_{m} {d}_{k , k + 1} - {\overline{d}}_{k , k + 1} )}^{2}}{{σ}^{2}} ) )}^{1 / K} \qquad ( 23 )$$

In this example, σ equals 4 mm and αₘ is a rescaling factor which is used to compensate for the size of a patient's jaw that may be larger or smaller than the average reference jaw. It is calculated to minimize the difference between the scaled distances and the reference distances:

$${α}_{m} = \frac{{\sum }_{k = 1}^{K - 1} {d}_{k , k + 1} {\tilde{d}}_{k , k + 1}}{{\sum }_{k = 1}^{K - 1} {d}_{k , k + 1}^{2}} \qquad ( 24 )$$

The value of the scaling factor am is preferably limited to be within an interval of [0.8, 1.2].

### Hypodontia Probability.

The previous sections always assume a full dentition as a reference for the investigated jaw, meaning that the reference configuration includes all possible tooth numbers. However, in hypodontia cases one or more teeth are missing without a physical gap between adjacent teeth. Accordingly, the average distances d̅_{k,k+1} in a case of hypodontia differ from regular cases of a missing tooth including a gap. This circumstance results in differing distance probabilities P_{dist}(Cₘ^{(K)}).

The different hypodontia cases investigated in this exemplary embodiment are a missing second incisor or a missing second premolar. These cases can occur on the right side, on the left side or on both sides of the jaw. Including the full dentition, this gives 7 different hypodontia configurations to investigate. Because hypodontia occurs less frequently than full dentition, a hypodontia probability P_{hypo}(Cₘ^{(K)}) is introduced in (20) which equals 1 for the full dentition case and 0.8 for the other 6 hypodontia cases.

### [Determining Crown Center Positions: Training the Neural Networks]

Like the anatomical landmarks, the crown center positions had been manually indicated in the 225 3D image datasets (FIG. 5: step 541). For each crown center position, a network was trained using 150 volume entities for each dataset. Each volume entity was randomly selected at a maximum distance of 12 voxels from each annotated crown center. The neural network was trained to estimate the offset vectors from volume entities towards a respective crown center in the same way as previously explained for the anatomical landmarks. To make all input data as similar as possible, intensity normalization and volume resampling was applied as previously discussed.

### [Estimate the Tooth Axes of the Detected Teeth]

With the dental situation of a patient analyzed for determining anatomical landmarks and crown center positions, there are further method steps that may be performed, in particular for eliminating manual input steps that previously had to be done by a dental clinician.

The previous sections explained how a tooth configuration for both the upper and the lower jaw can be calculated from 3D image data representing at least a part of a patient's dento-maxillofacial anatomy. These tooth configurations contain both the coordinates of the crown centers tⱼ together with their assigned tooth numbers j. In an additional step the principal tooth axis in an apical-coronal direction may be calculated.

In the following exemplary embodiment, this is done starting from a 2D axial z-slice in which the crown center ti is located and by tracking the corresponding voxel position throughout subsequent axial z-slices in the direction of the dental root. Preferably, a so-called Kanade-Lucas-Tomasi tracker is used with the adjacent axial z-slices acting as image frames. As already described above, the axial z-slice is a slice perpendicular to the axis of the voxel image volume that is closest to an apical-coronal direction. The original axes of the 3D image data and the voxel image volume may have been set by the scanner obtaining the 3D image data.

The path obtained by this tracker basically provides the basic principal tooth axis. For each axial slice the offset vector μ=(μx, μy) is searched which indicates the translation of the crown center (uₓ^{s}, u_{y}^{s}) in the current slice Z^{s}(x, y) with respect to the crown center (uₓ^{s-1}, u_{y}^{s-1}) in the previous slice Z^{s-1}(x, y). As already indicated above, the first (uₓ^{s}, u_{y}^{s}) is the determined crown center position (tₓ, t_{y}). This is done by minimizing the following objective function:

$$∈ ( μ ) = {\sum }_{x = {u}_{x}^{s - 1} - w}^{{u}_{x}^{s - 1} + w} {\sum }_{y = {u}_{y}^{s - 1} - w}^{{u}_{y}^{s - 1} + w} {[ {Z}^{s - 1} ( x , y ) - {Z}^{s} ( x + {μ}_{x} , y + {μ}_{y} ) ]}^{2} \qquad ( 25 )$$

Using this method, the center point of each tooth is tracked over a predetermined number of multiple slices (in this exemplary embodiment 24) in the direction from the occlusal plane to the roots. Next, the direction of the tooth is estimated by fitting a 3D line through the tracked points. Preferably, the line is represented by a starting point rₒ and a direction e:

$$r = {r}_{o} + γ e \qquad ( 33 )$$

To remove the influence of outliers, preferably a RANSAC procedure is used with, for example, 200 iterations. In each iteration, 3 random points are selected from the set of (uₓ^{s}, u_{y}^{s}, u_{z}^{s}) and a 3D line (in particular a linear 3D line) is fitted to these points.

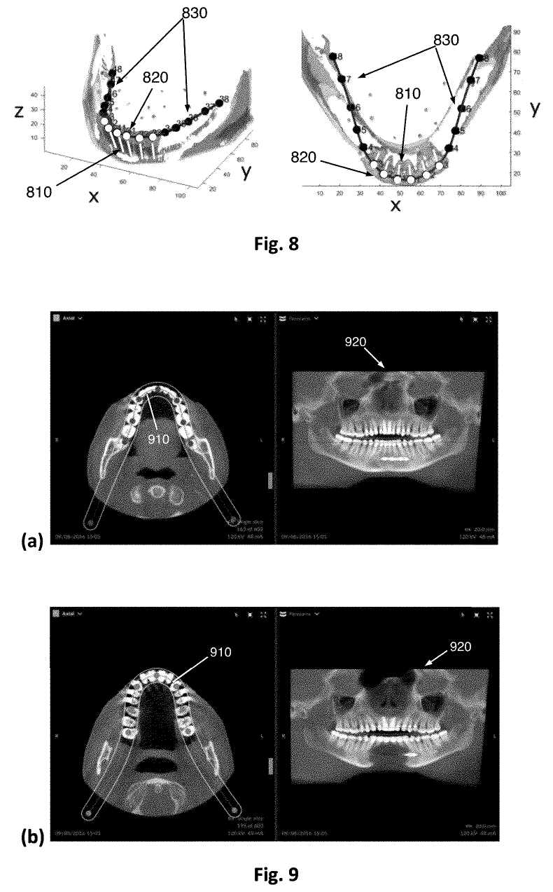

Next, all points that are located at a distance smaller than a predetermined inlier threshold, for example 1 mm, from this line are considered as inliers and the line is again fitted through these inliers. Once again, the number of inliers is calculated for this line. The iteration with the maximum number of inliers is considered as the best configuration, yielding the final 3D line representing the tooth axis. From this fitted line, particularly the direction may be used. The lines representing the tooth axis directions through the detected crown centers positions are depicted as white lines 810 for an exemplary case for the mandible of a patient in FIG. 8. In this figure, the crown center positions of teeth that have been found to be represented in the voxel image volume are depicted as white dots 820, whereas the estimated crown center positions of missing teeth are illustrated as black dots 830.

Alternatively, a tooth axis can be determined using a segmentation method. Tooth voxels in (CB)CT images are typically brighter than those of surrounding tissues and air due to a relatively high density of the tooth tissue. Using this property of tooth voxels, a rough segmentation of the tooth may be obtained from a detected crown center position by identifying the brighter voxels connected to the voxel comprising said crown center position.

Preferably, this segmentation is performed within a cropped image region. This cropped image region can be set from said detected crown center position by defining a volume surrounding said crown center, wherein the dimensions, shape, and position of said volume may be selected to narrowly encompass the tooth for which the crown center was detected. Subsequently a tooth axis can be determined for said segmented tooth, for instance using a principal component analysis.

In yet another embodiment, determining the tooth axis involves the use of template teeth. The template teeth may either be crown or full tooth (i.e. crown and root) models.

Based on the tooth label assigned to a given crown center position, a template tooth may be selected with a tooth type corresponding to that of the tooth represented at said crown center position. The selected template tooth may subsequently be aligned and/or fitted to said represented tooth.

Preferably, the template tooth and the represented tooth are aligned in an initial step by aligning said detected crown center position and the crown center position as determined for said template tooth. The tooth axis at said detected crown center position can then be derived from the tooth axis as determined for the aligned and/or fitted template tooth.

### [Add Missing Tooth Positions]

Once the crown center positions are detected together with their tooth number, the position of each tooth missing in the imaged patient dentition may also be determined. A crown center position of a missing tooth is preferably extrapolated based on (i) the crown center positions of the teeth represented in the 3D image data and associated tooth labels and (ii) information on the respective average crown center positions of the teeth (e.g. 32 teeth in case of an adult patient).

The average crown center positions may be determined from the training data by calculating an average crown center position and the variation observed from the average crown center position in the x, y and z direction for each of the 32 tooth numbers. An example is presented in FIG. 8, where the crown center positions are indicated with dots within the volumes of the imaged teeth. In this figure, the crown center positions of teeth that the patient misses but for which a crown center position has been estimated are also visualized by dots.

Accordingly and as illustrated in FIG. 8, a direction of a tooth axis may also be estimated for these missing teeth, preferably using a linear interpolation based on the tooth axis directions of neighboring teeth. If the missing teeth has only one neighboring tooth, a linear interpolation between tooth axes of neighboring teeth cannot be performed. Instead, the direction of the neighboring tooth is copied.

### [Estimating Dental Planes]

The detected crown center positions in the upper and lower jaw allow determining the dental planes of the upper and the lower jaw, respectively. The dental plane of a jaw is estimated by fitting a plane through the crown center positions as determined for that jaw. Preferably, the crown center positions of the wisdom teeth are not included in this fitting process.

If both the upper and the lower jaw contain teeth, an averaged dental plane can be fitted through the total set of crown centers positions from both the upper and the lower jaw. Preferably, the crown center positions of the wisdom teeth are not considered when fitting the average dental plane to the crown center positions.

After determining the upper, lower and/or average dental plane, a dental transformation D can be determined for either one of said dental planes, wherein said transformation transforms the original volume image with the dental plane parallel to the Z-plane of the volume image. Preferably, this dental transformation also orients the imaged skull anatomy with the incisors facing to the front and with the incisors in the origin.

Applying said dental transformation D to the dental crown center positions detected in an upper or lower jaw provides corresponding crown positions in a 2D planar reslice plane. From the (x,y) components of these transformed crown centers position a 2D spline curve can be calculated located within said dental plane. In this way, 2D dental curves can be determined for the upper and lower jaw, respectively.

### [Generating Reslices]

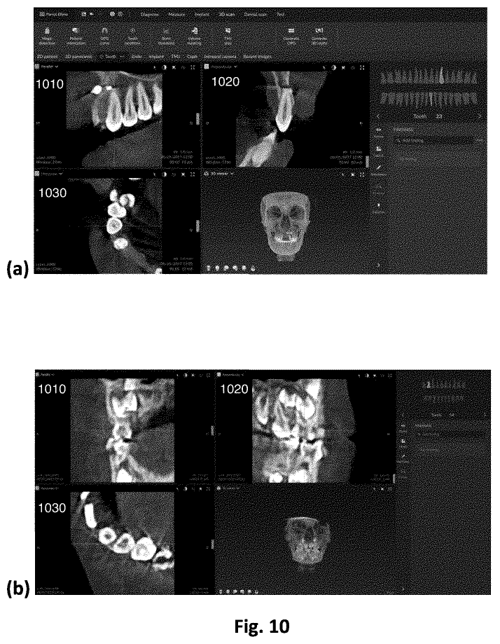

Said 2D or 3D dental curves (FIG. 9: reference sign 910) allow defining a curved reslice plane through the voxel image volume suitable for projecting a panoramic reslice image 920 thereon.

Determining the tooth axis may further serve as a reference for generating a mesial-distal reslice image 1010, a buccal/lingual-oral reslice image 1020 and/or an axial reslice image 1030 at a given or selected tooth position (see FIG. 10). The mesial-distal reslice image 1010 comprises the tooth axis and is essentially parallel to a 2D or 3D dental curve at the selected tooth position. The buccal/lingual-oral reslice image 1020 also comprises the tooth axis but is essentially perpendicular to a 2D or 3D dental curve at said tooth position. The axial reslice image 1030 comprises the detected or possible crown center position at said tooth position, wherein the plane of the axial reslice is perpendicular to the tooth axis.

### [Generating a Tooth Chart Using Said Detected Crown Center Positions]

The detected crown center positions and associated tooth labels may further allow for automatically populating a patient specific tooth chart with icons indicating the presence or absence of a tooth behind a given tooth number in a patient's dentition. Optionally, said tooth chart may further reflect the relative positioning of the patient's teeth based on the distances between the detected crown center positions.

### [Using a Tooth Chart to Navigate Volumetric Image Data and to Generate Tooth Specific Reslices]

The computer implemented method may be integrated as a computer program product. It may be implemented as a software application comprising a user interface displaying a tooth chart comprising selectable icons denoting tooth positions. When a user selects one of said icons, the application may automatically generate and display said mesial-distal, the buccal/lingual-oral and/or the axial reslice image for said selected tooth position as derived from the volumetric 3D image data representing the patient's dento-maxillofacial anatomy. In an embodiment, the application may display said volumetric 3D image data and cause said 3D image to be automatically oriented in the display window so that the selected tooth position is visible.

1. A computer implemented method for automatically generating and displaying, derived from 3D image data, a 2D image of a portion of a patient's maxillofacial anatomy, said method comprising the steps:
receiving 3D image data comprising a voxel image volume representing the maxillofacial anatomy of the patient, the voxel image volume including teeth of the patient and each voxel of the voxel image volume being associated with a radiation intensity value,
detecting one or more anatomical landmarks in said voxel image volume using a first artificial neural network,
using a second artificial neural network for determining a crown center position for each tooth of multiple teeth included in the voxel image volume with the detected anatomical landmarks as reference points, and
generating the 2D image as a reslice image from the voxel image volume based on at least one crown center position.

2. The method according to claim 1, further comprising the step of calculating a curved path following a dental arch of the patient's teeth using said one or more anatomical landmarks and/or by fitting a curve to the determined crown center positions and using the curved path as a reference for generating the reslice image.

3. The method according to claim 2, the method further comprising the step of detecting the center of the left and/or right condyle head using the first artificial neural network, wherein the curved path following the dental arch is calculated using the center of the left and/or right condyle head as endpoints.

4. The method according to claim 2, wherein the reslice image is a panoramic image generated based on said curved path following the arch form of the patient's teeth.

5. The method according to claim 1, wherein method further comprises the step of analyzing the 3D image data within at least an image region surrounding the determined crown center position of one of the teeth to derive a tooth axis, wherein the tooth axis intersects a coronal crown surface of the tooth, extends from this intersection towards an apical end of the tooth, and comprises the crown center position determined for the tooth.

6. The method according to claim 5, wherein deriving the tooth axis comprises, starting from a cross-section of the tooth including the estimated crown center position, the step of tracking a corresponding position in a next adjacent cross-section and proceeding with tracking corresponding positions in adjacent cross-sections along the tooth, in particular in a direction towards the apical side of the tooth, and the step of calculating the tooth axis based on these corresponding positions.

7. The method according to claim 5, wherein the orientation of the derived tooth axis is calculated relative to the curved path following the dental arch.

8. The method according to claim 5, wherein the reslice image is generated and displayed as a cross-sectional reslice image comprising said crown center, the plane of the cross-sectional reslice image being perpendicular to the tooth axis of the tooth.

9. The method according to claim 2, wherein the reslice image is generated and displayed as a mesial-distal reslice image that comprises the tooth axis and is essentially oriented parallel to the curved path at the position of one of the teeth or a buccal/labial-oral reslice image that comprises the tooth axis and is essentially oriented perpendicular to the curved path at the position of one of the teeth.

10. The method according to claim 1, wherein the second artificial neural network comprises a module trained to estimate the three components of an offset vector from a position within said voxel image volume towards a predicted crown center position.

11. The method according to claim 2, wherein determining the crown center positions further comprises assigning a tooth label for each determined crown center position using said detected anatomical landmarks and by analyzing the relative positions of said crown center positions.

12. The method according to claim 11, further comprising the step of deriving a virtual crown center position and an associated virtual tooth label for any tooth missing in the 3D image data of the patient from said determined crown center positions and assigned tooth labels.

13. The method according to claim 12, further comprising the step of generating and displaying a tooth chart comprising selectable icons denoting teeth represented in the 3D image data and/or any tooth missing in the 3D image data, wherein upon selection of one of the icons a 2D image of the tooth of the selected icon is displayed and includes at least the reslice image.

14. The method according to claim 12, wherein the method further comprises displaying the 3D image data and displaying a marker at the position of each determined crown center position and/or each virtual crown center position.

15. The method according to claim 12, further comprising the steps:
extrapolating a virtual tooth axis for the at least one missing tooth, wherein the virtual tooth axis comprises the virtual crown center position of the missing tooth, and
calculating a direction of the virtual tooth axis based on a local direction of the curved path following the dental arch and/or the direction of a tooth axis of at least one neighboring tooth.

16. The method according to claim 12, wherein calculating the curved path following the dental arch of the patient's teeth comprises fitting the curve of the path to both the determined crown center positions of the teeth represented in the 3D image data and each virtual crown center position.

17. The method according to claim 15, further comprising the step of generating and displaying a reslice image of a reslice plane comprising the virtual tooth axis of the missing tooth.

18. The method according to claim 15, wherein the method further comprises displaying the 3D image data and displaying for each crown center position a line corresponding to the tooth axis and/or for each virtual crown center position a line corresponding to the virtual tooth axis.

19. A computer implemented method for automatically generating and displaying, derived from 3D image data, a 2D image of a portion of a patient's maxillofacial anatomy, said method comprising the steps:
receiving 3D image data comprising a voxel image volume representing the maxillofacial anatomy of the patient, the voxel image volume including teeth of the patient and each voxel of the voxel image volume being associated with a radiation intensity value,
detecting one or more anatomical landmarks in said voxel image volume using a first artificial neural network,
using a second artificial neural network for determining a crown center position for each tooth of multiple teeth included in the voxel image volume with the detected anatomical landmarks as reference points, and
generating the 2D image as a reslice image from the voxel image volume based on at least one crown center position;
calculating a curved path following a dental arch of the patient's teeth using said one or more anatomical landmarks and/or by fitting a curve to the determined crown center positions and using the curved path as a reference for generating the reslice image;
detecting the center of the left and/or right condyle head using the first artificial neural network, wherein the curved path following the dental arch is calculated using the center of the left and/or right condyle head as endpoints,
wherein the reslice image is a panoramic image generated based on said curved path following the arch form of the patient's teeth.

20. A computer implemented method for automatically generating and displaying, derived from 3D image data, a 2D image of a portion of a patient's maxillofacial anatomy, said method comprising the steps:
receiving 3D image data comprising a voxel image volume representing the maxillofacial anatomy of the patient, the voxel image volume including teeth of the patient and each voxel of the voxel image volume being associated with a radiation intensity value,
detecting one or more anatomical landmarks in said voxel image volume using a first artificial neural network,
using a second artificial neural network for determining a crown center position for each tooth of multiple teeth included in the voxel image volume with the detected anatomical landmarks as reference points;
generating the 2D image as a reslice image from the voxel image volume based on at least one crown center position;
calculating a curved path following a dental arch of the patient's teeth using said one or more anatomical landmarks and/or by fitting a curve to the determined crown center positions and using the curved path as a reference for generating the reslice image; and
analyzing the 3D image data within at least an image region surrounding the determined crown center position of one of the teeth to derive a tooth axis, wherein the tooth axis intersects a coronal crown surface of the tooth, extends from this intersection towards an apical end of the tooth, and comprises the crown center position determined for the tooth,
wherein the reslice image is generated and displayed as a cross-sectional reslice image comprising said crown center, the plane of the cross-sectional reslice image being perpendicular to the tooth axis of the tooth, or
wherein the reslice image is generated and displayed as a mesial-distal reslice image that comprises the tooth axis and is essentially oriented parallel to the curved path at the position of one of the teeth or a buccal/labial-oral reslice image that comprises the tooth axis and is essentially oriented perpendicular to the curved path at the position of one of the teeth.
